(19) United States
(12) Patent Application Publication
(10) Pub. No.: US 2026/0165600 A1
(43) Pub. Date: Jun. 18, 2026

(54) ANIMAL HEALTH MONITORING SYSTEM

(71) Applicant: EQUIMETRICS LIMITED, Co. Kilkenny (IE)

(72) Inventors: Annemarie O'BRIEN, Co. Kilkenny (IE); Tom CURTIS, Co. Kilkenny (IE)

(73) Assignee: EQUIMETRICS LIMITED, Co. Kilkenny (IE)

(21) Appl. No.: 19/128,842

(22) PCT Filed: Nov. 9, 2023
(86) PCT No.: PCT/EP2023/081296

§ 371 Date: May 9, 2025

(30) Foreign Application Priority Data
Nov. 10, 2022 (GB) 2216799.3
Nov. 10, 2022 (GB) 2216813.2

(51) Int. Cl. A61B 5/08; A61B 5/0205; A61B 5/282

(52) U.S. Cl. CPC A61B 5/0816; A61B 5/0205; A61B 5/282

(57) ABSTRACT

An animal health monitoring system comprising a chest harness having a chest portion mountable at the chest of the animal and a strap for supporting the chest portion on the animal, and a rib portion attached to the chest portion configured for positioning over the ribs of the animal in which the rib portion comprises a sixth rib radius locus for receiving an ECG sensor configured to locate adjacent a sixth rib radius of the animal.

### FIELD OF INVENTION

This invention relates to an animal health monitoring system for remotely monitoring health parameters of an animal such as a horse, dromedary or dog.

### BACKGROUND OF THE INVENTION

Animal health and wellbeing determine performance in competition and are of constant concern and interest both to owners and trainers. For example, four of the critical animal health parameters that determine overall health and wellbeing of the horse are temperature, heart rate, respiration, and moisture (sweating). A fifth parameter which can provide key information on the wellbeing of an animal is movement where abnormal movement/behaviour can be indicative of potential health problems. The ability to assess animal health parameters can be exceedingly valuable to owners, vets, trainers and the like.

Harnesses and similar devices are known for monitoring animal health parameters. For example, it is known to use equine straps and the like fitted with sensors to monitor some health parameters. However, the known devices are cumbersome to use and can have exposed wiring and the like which can present safety risks to horse and rider and which can be easily damaged resulting in poor data harvesting.

In addition, it can be difficult to position the sensors employed in known devices correctly on the animal for optimal signal generation. In particular, it can be difficult to optimally, correctly and accurately locate and maintain ECG sensors in the correct position on the animal to harvest reliable ECG data. In addition, the necessary wiring associated with known ECG equipment can easily become damaged in use and present a safety risk to both the horse and rider. In addition, wiring can give rise to noise interference which can compromise the ECG signal.

WO 2018/144972 A1, WO 2019/235362 A1, US 2020/0359605 A1, WO 2018/143461 A1, WO 2018/002705 A1 and WO 2006/113804 A1 describe various monitoring systems. However, all of the documents describe systems which are still prone to error as they fails to describe health monitoring systems in which the device automatically locates a sixth rib radius locus with ease.

An object of the invention is to overcome at least some of the problems of the prior art.

### SUMMARY OF THE INVENTION

In its broadest sense, the invention relates to an animal health monitoring system comprising:
a chest harness having a chest portion, which can be a chest pad, mountable at the chest of the animal and a strap for supporting the chest portion on the animal,
and a rib portion, which can be a rib pad, attached to the chest portion/pad configured for positioning at the ribs of the animal
wherein the rib portion/pad and/or the chest portion/pad comprises a sixth rib radius locus for receiving an ECG sensor configured to locate adjacent a sixth rib radius of the animal.

According to the invention there is provided 1. An animal health monitoring system comprising:
a chest harness having a chest portion mountable at the chest of the animal and a strap for supporting the chest portion on the animal,
and a rib portion attached to the chest portion configured for positioning at the ribs of the animal
wherein the rib portion and/or the chest portion comprises a sixth rib radius locus for receiving an ECG sensor configured to locate the ECG locus adjacent a sixth rib radius of the animal.

In one embodiment, at least a first ECG sensor is located at the sixth rib radius locus.

In one embodiment, the ECG electrode is detachably mounted on the sixth rib radius locus with a connector.

In one embodiment, the rib portion and/or the chest portion comprise oppositely disposed sixth rib radius loci for receiving first and second ECG sensors.

In one embodiment, the rib portion and/or chest portion comprises a third ECG reference sensor at a sixth rib radius locus.

In one embodiment, the ECG sensors comprise ECG electrodes mounted on an external animal contacting surface of the sixth rib radius loci.

In one embodiment, the system further comprises an electronic assembly communicable with the ECG sensor mounted at a mounting on the chest portion.

In one embodiment, the system further comprises integrated wiring concealed within the rib portion and the chest portion extending between the sensor and the electronic assembly.

In one embodiment, the wiring is threaded between the sensor and the electronic assembly in an undulating, coiled or zig-zag pattern to minimise mechanical stress on the wiring.

In one embodiment, the wiring is concealed within an inner layer of the chest portion and rib portion.

In one embodiment, the inner layer comprises an air mesh fabric.

In one embodiment, the wiring is connected to the electronic assembly via a magnetic connector at the mounting.

In one embodiment, the magnetic connector is a nine pin magnetic connector.

In one embodiment, the magnetic connector comprises a fluid-tight magnetic connector.

In one embodiment, the mounting comprises a holster for housing the electronic assembly.

In one embodiment, the holster is centrally located on the chest portion.

In one embodiment, the electronic assembly comprises a self-contained and removable cassette electronic assembly complementary in size and shape with the holster.

In one embodiment, the cassette comprises a male or female part of the magnetic connector and the holster comprises a complementary male or female part of the magnetic connector.

In one embodiment, the wiring comprises low-noise wires.

In one embodiment, the wiring is connected to the ECG sensor by a crimped connector.

In one embodiment, the system comprises a tough fabric outer layer.

In one embodiment, the harness comprises an ergonomic wither pad connecting the chest portion with the rib portion.

In one embodiment, the wither pad is connectable by straps to the rib portion and the chest portion and is provided with strap mountings shaped and configured in accordance with the shoulder anatomy of the animal.

In one embodiment, the wither pad comprises a spinal clearance or a spine recess to accommodate the spine of the animal.

In one embodiment, the wither pad comprises a grip fabric on its animal contacting side.

In one embodiment, the system further comprises a sensor which can be any or all of a temperature, respiration, moisture, GPS and movement and heartrate sensor.

In one embodiment, the chest harness comprises a central panel which extends between the rib portion and the chest portion.

In one embodiment the system further comprises a saddle pad for ambulatory ECG measurement of the animal, the saddle pad comprising:
a spine portion;
first and second side aprons having a front end and a rear end extending from the spine portion configured for positioning over the ribs of the animal, at least one of the side aprons comprising a sixth rib radius portion for receiving an ECG sensor configured to locate adjacent a sixth rib radius of the animal,
integrated wiring concealed within the saddle pad extending between the sensor and an electronic assembly
wherein the self-contained and removable cassette electronic assembly is also mountable at a saddle pad holster to also serve as the electronic assembly for the saddle pad.

In one embodiment, the animal health monitoring system further comprises a saddle pad for ambulatory ECG measurement of an animal, the saddle pad comprising:
a spine portion, and
first and second side aprons having a front end and a rear end extending from the spine portion configured for positioning over the ribs of the animal wherein at least one of the side aprons comprises a sixth rib radius portion defining an ECG locus for receiving an ECG sensor configured to locate the ECG locus adjacent a sixth rib radius of the animal
integrated wiring concealed within the saddle pad extending between the sensor and an electronic assembly
wherein the self-contained and removable cassette electronic assembly is also mountable at a saddle pad holster to also serve as the electronic assembly for the saddle pad.

In any embodiment, the system is an equine health monitoring system.

In any embodiment of the saddle pad of the system of the invention, the sixth rib radius portion is configured to be located towards the upper side of the sixth rib radius.

In one embodiment, at least a first ECG sensor is located at the ECG locus on the sixth rib radius portion.

In one embodiment, the ECG electrode is detachably mounted on the ECG locus on the sixth rib radius portion with a connector.

In one embodiment, the sixth rib radius portion projects convexly outwards from the side apron.

In one embodiment, the sixth rib radius portion comprises a flap.

In any embodiment, the sixth rib radius portion is spaced between the front and rear end of the side apron to coincide with a girth of a saddle so that the saddle pad is attachable to an animal by sandwiching the sixth rib radius portion between the sixth rib radius of the animal and the girth.

In one embodiment, the first side apron comprises a first sixth rib radius portion and the second side apron comprises a second sixth rib radius portion.

In one embodiment, the first or second side apron comprises up to three ECG sensors.

In one embodiment, the first sixth rib radius portion comprises a first ECG sensor and the second sixth rib radius portion comprises a second ECG sensor.

In one embodiment, the first sixth rib radius portion or the second sixth rib radius portion comprises a third ECG reference sensor.

In one embodiment, the saddle pad is a multi-layer saddle pad comprising an outer layer, an inner layer, an ECG sensor carrier layer and an ECG compressor layer (510) for urging ECG sensors against an animal.

In one embodiment, the ECG compressor layer comprises compressor blocks for the sensors.

In one embodiment, the inner layer comprises windows for the compressor blocks.

In any embodiment, the saddle pad comprises natural materials.

In one embodiment, the inner layer comprises an air mesh fabric.

In one embodiment, the saddle pad comprises a ripstop fabric outer layer.

In any embodiment, the ECG sensors comprise ECG electrodes.

In one embodiment, the ECG electrodes have an increased surface area to cover a greater area of an animal's ribs.

In one embodiment, the ECG electrodes comprise carbon silicone rubber electrodes.

In one embodiment, the saddle pad comprises additional health sensors.

In one embodiment, the additional health sensor is a GPS sensor, a gyroscope, an accelerometers and/or a temperature sensor.

In one embodiment, the saddle pad further comprises an electronic assembly communicable with the ECG sensor mounted at a mounting towards the rear end of the first or second side apron.

In one embodiment, the electronic assembly is disposed forwardly of the saddle pad rear end towards the saddle pad front end 100 to be as proximate to the ECG electrodes.

In one embodiment, the electronic assembly is located towards the ECG loci at the sixth rib radius portion.

In one embodiment, the saddle pad further comprises integrated wiring concealed within the saddle pad extending between the sensor and the electronic assembly.

In any embodiment, the wiring is configured to extend rearwardly from the electronic assembly away from a saddle in use.

In one embodiment, the wiring is threaded between the sensor and the electronic assembly in an undulating, coiled or zig-zag pattern to minimise mechanical stress on the wiring.

In one embodiment, the wiring is electronically connected to the electronic assembly via a magnetic connector at the mounting.

In one embodiment, the magnetic connector is a nine pin magnetic connector.

In one embodiment, the magnetic connector comprises a fluid-tight magnetic connector.

In any embodiment, the saddle pad further comprises a PCB in which the PCB comprises potting.

In one embodiment, the mounting comprises a holster for housing the electronic assembly.

In one embodiment, the electronic assembly comprises a self-contained and removable cassette electronic assembly complementary in size and shape with the holster.

In one embodiment, the cassette comprises a male or female part of the magnetic connector and the holster comprises a complementary male or female part of the magnetic connector.

In one embodiment, the wiring comprises low-noise wires.

In one embodiment, the wiring is connected to the ECG sensor by a crimped connector.

The invention also extends to a method ECG measurement of an animal comprising:
mounting a heath monitoring system as hereinbefore defined on the animal and recording the detected ECG data.

In one embodiment, the method further comprises displaying the ECG data.

In one embodiment, the method further comprises first scanning the animal's microchip to record the animal's Unique Identifier Code (UIC).

In one embodiment, the method further comprises wirelessly transmitting the UIC to a user.

In one embodiment, the method further comprises wirelessly transmitting the ECG data to the user and synchronising the ECG data with the UIC.

In one embodiment, the wireless transmission occurs to an app.

Four of the critical animal health parameters that determine overall health and wellbeing of the horse are temperature, heart rate, respiration, and moisture (sweating). The health monitoring system of the invention automatically locates the ECG loci adjacent the sixth rib radius of an animal and can measures all of the above data sets in a smart wearable chest harness application device, which houses safely, all wiring for a multitude of sensors and electrode pads which lead to and finish at the holster device on the front chest. This holster device holds the electronic assembly which receives the data from the horse, processes it and uploads it to the cloud for processing to the user interface. A fifth measurement namely movement which incorporates standing, walking, lying, rolling and the prone position of the horse is measured using motion sensors namely accelerometers which are also incorporated into the electronic assembly. This information is analysed on the device using smart algorithms and the data is then transmitted to the cloud using one of the communication protocols as outlined below. The information is then transmitted to the end user's smart device where abnormal behaviour can be flagged and an alert sent where necessary. Alerts can also be turned on to notify the end user when the horse's data demonstrates abnormal behaviour or when pre-set thresholds have been breached.

The health monitoring system can also include a saddle pad for ambulatory ECG measurement of the animal which is complementary with the harness in that the saddle pad also has sixth rib radius portions defining ECG loci and a holster like that of the harness that can receive the same electronic assembly as the harness so that the electronic assembly is interchangeable between the harness and the saddle pad as required in health monitoring system of the invention.

Accordingly, the health monitoring system of the invention is a universal health monitoring system that can be used to monitor animals at rest (the harness) and during movement, e.g. whilst racing or training, (the saddle pad) with a single electronic assembly that is interchangeable between the harness and the saddle pad.

In the following description, the invention is often described in relation to a horse. However, the invention should not be construed as being limited to equine applications as, as will be appreciated by those skilled in the art, the invention can be employed with a wide range of other animals such as dromedaries and the like.

### Electronic Assembly

The interchangeable electronic assembly is designed to communicate securely with external communications and analytic hubs. The chosen communications method employed can be selected from any of the following namely, Wi-Fi, GSM, Low power Wi-Fi, LoRa, Bluetooth or combinations of the above. The electronic assembly can read the animal's identity chip (Unique Identifier Code (UIC)) in advance of deployment to ensure correct animal selection for investigation and avoid costly errors or misinformation.

The signals captured from the electronic assembly are transmitted to a communications hub where proprietary software and algorithms present the necessary data for decision making in a user friendly and visual form on a laptop or mobile device. The information presented is compared and contrasted with normal data from the individual animal so as to demonstrate variances or divergence over time and so alert owner or management in real time.

### Chest Harness

The chest harness has been designed to provide the ideal application device for multiple sensors, electronics & electrode pads for use with horses, in a safe manner. The chest harness has been designed in this manner for an animal such as a horse to safely wear the device for extended periods at rest, post exercise, in the stable, in the field, in transport in trailers, trucks and aeroplanes. ‘At Rest’ can be defined as the horse not exerting itself in sport, being ridden or exercised and when appropriate being left to its own devices in a stable environment or in a paddock.

Importantly, the harness also incorporates cardiac ECG capability, within the electronic assembly, and has strategically placed electrodes placed circa the sixth rib radius on the left of the horse, which in one embodiment can utilise a second and third electrode positioned on the right shoulder area of the horse-to ensure a good cross sectional reading of the heart whilst the horse is at rest. This has been done specifically to enhance signal morphology and aid diagnostic accuracy in the horse at rest. The purpose of three electrodes is that you will achieve a good cross section reading of the horse's heart (from the electrode positioned at the sixth rib area on the left across to the one on the opposite side of the animal, located on the right shoulder). The third electrode is also placed on the right shoulder side of the animal and just above the ECG reading electrode as a reference grounding lead.

### Microchip Reader

The vast majority of equines can be identified by a unique embedded microchip that is inserted into the neck of every foal when it's born at approximately 3 months of age. The system of the invention contains a microchip reader that has been specially designed to read the UIC of the horse's embedded microchip easily and quickly. The device includes a specific coil length and shape that reads the embedded microchip at an effective distance. This design is critical to the efficient use of identifying the horse very quickly and allowing each horse's biometric data to be stored under the UIC.

### Location

The device can contain a GPS sensor which identifies where the horse is located in real time, the GPS locator provides this data by the use of satellites to give exact location coordinates.

### Management Sensors

The system of the invention can include humidity, temperature and moisture sensors, etc. to allow for the interrogation of any warranty failings, problems or device issues remotely.

### Holster and Electronic Assembly Connection

The wires from the electrodes are specifically chosen for their shielded low tribo noise capability which can be threaded through an air mesh padded fabric to minimise mechanical stress and reduce interference artefacts. Air circulation, space for wiring and padding are absolutely necessary for the harness to be breathable, secure and allow for movement whilst protecting the horse from rubbing, skin irritation or pressure points. The_wires travel throughout the harness to then terminate at a nine-pin magnetic connection which connects to the electronic assembly to process, store and feed data to the cloud. The specific nine pin connector enjoys a number of advantages:
1. Gold plating to reduce risk of corrosion from the elements/harsh environment and of course proximity to sweat of the horse;
2. 9 Pins to service 3 for the ECG, 2 for Galvanic Skin Response (Bioimpedance) 2 for data & 2 for power;
3. Magnetic—serves two purposes. A. for correct mating of male to female connection point in the holster (which is unforgiving should the mating of the electronic assembly to the holster on the harness application device be incorrect) B. This is unforgiving in nature due to the safety and security required to keep the electronic assembly safely in situ whilst the horse moves, lies down, rolls etc. The electronic assembly is also securely held in place with the addition of 2 push clips on either side of the assembly;
4. Sealed/Waterproof grading—the specific connectors chosen are sealed—thus limits the risk of moisture ingress to either the electronics in the holster and the electronic assembly thus limiting the risk of water ingress when the devices are not connected and vulnerable.

Accordingly, the nine-pin gold plated magnetic connector utilised in the design process addresses specific needs of the system. The male side of the connector is connected to the electronic assembly and the female side of the connector is incorporated into the holster on the harness. It is clear to see in these drawings that the connector is a sealed connector, which when adequately sealed within the holster housing with potting compound and housed within the electronic assembly housing-maintains high levels of waterproofness demanded by the product.

This specific connection aids in ensuring perfect conductivity for the ECG signal extraction and secure fitting of the electronic assembly to a live and moving animal. The signal and data generated is captured by the electronic assembly which is connected to the internally wired system and attached externally to a magnetic connector block encapsulated in the integrated holster. The electronic assembly can communicate directly by Bluetooth or other enabled communication with an App and then data be forwarded to an analytical structure and subsequently through a series of algorithms to the cloud. The final output is then displayed on a device of choice either handheld or other and preferably displays a graph of normal or previous ECG traces versus the current extraction. This enables a direct observation for vets or trainers and horse owners to any changes or abnormalities in the animal's wellbeing and behaviour.

### Material Selection

Heating and animal sweat including fabric stretching and compression present severe design constraints and survival risks. The sheer nature of a horse's movement, rolling lying down, rubbing against stable walls, scratching etc. has significant impact on the materials chosen. The materials of the system of the invention are of significant important to reduce interference in the ECG signal/readings, wiring must be of appropriate materials and insulated to overcome this issue and the materials themselves must not contain high levels of interference enhancing fibres. The materials chosen, including the air mesh fabric, allow for stretching and movement whilst also ensuring the wiring is protected from movement, sweat and all materials chosen have proven to be safe for a horse to wear safely unsupervised. Movement/slipping of the of the chest harness is also a consideration as is potential damage to any wiring during use, as such a tough outer layer with incorporated stretch capability is provided for the purpose to ensure that the device and wiring are protected sufficiently in the harsh environment to which it will be placed. A suitable tough outer fabric layer is a 600-denier outer fabric with high stretch capacity to allow for maximum comfort, breathability for the horse, but tough to enough to withstand the environment. This combined with an appropriate grip material on the underside of the wither pad eliminates the risk of the harness slipping and moving—as this is a key anchoring point in the design. The wither is a delicate and sensitive point in the horse's anatomy and the wither pad accounts for this by providing sufficient wither/spinal clearance in its design. A structured spinal clearance is incorporated into the wither pad design, grip can be placed on either side of this clearance to prevent movement/slipping.

The chest harness and saddle pad can share materials of construction which include multiple mesh and foams in order to avoid overheating/sweating and allow for stretching. These also provide protection to the wiring integrity which can be both coiled and zig zagged to compensate for movement. Stitching patterns are carefully designed so as to avoid contact with the internal wiring. As indicated above, the external and outer layers can be strong stretch tough layers of water-resistant materials and provide shape retention during use. The saddle pad can be formed from natural materials where possible to minimize sweating.

### Measurement/Assessment Components

Heartrate and respiration can be measured in two ways, one by the extraction of the data from the ECG and secondly by extracting the data from accelerometer and movement sensors.

Heart health and assessment can be determined by the ECG feature incorporated into the electronic device and with the necessary sensors placed strategically so as to get the optimum signal output from the coronary area. ECG is considered gold standard for establishing cardiac arrythmias and anomalies.

The horse's respiration rate can also be derived from the ECG reading. Both of these measurements are feasible because of the location of the sensor arrays in the harness placed strategically in a secured location on the animal's lower chest/torso and in proximity of the 6^{th} rib area radius. The ECG is affected by the strategic location of three contact electrodes in the harness fabric connected to the electronic assembly.

The temperature measurement is enabled by an infra-red aperture in the harness assembly.

Skin moisture content can be measured by placing two metal electrodes at a preset distance apart and located on the harness while connected by internal wiring to the electronic assembly. The animal sweat is electrically conductive and correlates with animal wellbeing, so a determination of the micro current is a proxy for health and wellbeing.

Movement is a key measurable to examine in equine wellbeing. Excessive movement, when the animal should be at rest, can and frequently does indicate that there may be a problem or upset. A horse rolling excessively or walking in circles in their stable can be indicative of pain & upset for example, as such—the horse would warrant veterinary or human assessment to ensure wellbeing. Movement is measured in our device using accelerometers and gyroscopes.

These five fundamental parameter measurements are made feasible by the incorporation of the necessary electronic sensors and electrodes in the internally wired harness and electronic assembly placed in the most advantageous location on the animal's chest. This placement also minimizes any risk of damage by the animal to the harness while housed or mobile.

### Holster for Electronic Assembly

The electronic assembly is securely and strategically placed in a strong weatherproof holster from which it can be easily removed or replaced and is located on the animal's lower chest in the closest proximity to the organs of interest namely heart and lungs. Electronic wiring from the holster to the various sensors/electrodes is achieved by incorporating them in the fabric of the harness and securely protecting them against damage or interference. The holster has magnetic contact terminals in its inner base which mate with the removable electronic assembly when inserted. The electronic assembly contains motion (inertia) sensors, pressure sensors, global positioning sensors, RFID reader, and others as required such as environmental.

The electronic assembly is designed to communicate securely with external communications and analytic hubs. The chosen communications method employed can be selected from any of the following namely, Wi-Fi, GSM, Low power Wi-Fi, LoRa, Bluetooth or combinations of the above. The electronic assembly will read the animals identity chip in advance of deployment to ensure correct animal selection for investigation and avoid costly errors.

The signals captured from the electronic assembly are transmitted to a communications hub where proprietary software and algorithms present the necessary data for decision making in a user friendly and visual form on a laptop or mobile device. The information presented is compared and contrasted with normal data from the individual animal so as to demonstrate variances or divergence over time and so alert owner or management in real time.

In summary, the chest harness of the system of the invention is a complex structure in that it accomplishes a stable architecture both to house the many electronic components and also to maintain the sensors and ECG electrodes in the required and preferential location on the animal's chest. The primary support is by way of the ergonomic wither pad, located on the animal's back from which the adjustable straps are attached. This allows for adjustment to fit the chest harness to the exact position required for optimum performance. These straps can be manufactured from a combination of functional materials (natural/elastic/air mesh/ripstop) as required and are weather resistant and robust. Descending from these straps is the main harness structure which comprises electronic wiring embedded in a fabric manufactured from air mesh, ripstop and electronics. The ECG electrodes and the resistive electrodes are permanently incorporated in this fabric and are located at the precise locations and distances for optimum signal capture. The harness is further supported in place by the central panel which extends through the front legs and secures around the girth area ensuring a safe and secure placement which does not interfere with movement. The positioning of the electronic assembly at the chest is strategic in its placement to avoid failure or damage by the horse as it is one of the few parts on a horse where the horse is unable to reach and therefore cannot interfere with or damage the device. The chest harness also houses the holster through which temperature measurements can be effected with an infrared device.

### Saddle Pad

Like the harness, the saddle pad of the system serves a dual function as the carrier and support for the location of the ECG sensors in contact with an animal's torso which are automatically correctly positioned relative to the rib cage for a clear cardiac reading. The saddle pad is shaped and contoured with the sixth rib radius portion to securely and accurately automatically locate integrated ECG sensors/electrodes adjacent and more particularly at the sixth rib radius to enhance signal morphology and aid diagnostic accuracy.

A saddle pad of the system of the invention is described in detail in our co-pending PCT patent application of even date, the contents of which are incorporated herein by reference. As discussed further below, the saddle pad is for ambulatory ECG measurement of an animal, and comprises a spine portion, and first and second side aprons having a front end and a rear end extending from the spine portion configured for positioning over the ribs of the animal wherein at least one of the side aprons comprises a sixth rib radius portion defining an ECG locus for receiving an ECG sensor configured to locate the ECG locus adjacent a sixth rib radius of the animal. Importantly, as indicated above, the saddle pad comprises an electronic assembly which is the same electronic assembly employed with the harness which is communicable with the ECG sensor loci and a holster similar to the harness holster for housing the cassette type electronic assembly i.e. the same cassette type electronic assembly is interchangeable between the harness and the saddle pad.

In the system of the invention, wires from the electrodes are specifically chosen for their shielded low tribo noise capability and can be threaded through the fabric in a zig-zag pattern to minimise mechanical stress and reduce interference artefacts. The air circulation, space for wiring and padding functions provided by air mesh fabric, where employed, ensure that the system protects an animal whilst housing and concealing the necessary wiring safely to prevent injury to the animal and rider where present. The air mesh fabric also protects the wiring from damage to ensure secure and uninterrupted communication between the ECG sensor and associated electronic assembly.

The structure and configuration of the system of the invention accounts for the natural movement of an animal. The construction of the rib, chest and saddle pads of the invention and the wiring and fluid-tight magnetic connector at the holsters on the harness and saddle pad ensure that sensor data harvesting is not compromised by body heat and animal sweat. The fabrics and structures employed resist fabric stretching and by housing the concealed wiring eliminate safety risks.

The system of the invention therefore provides a solution to vets, trainers, owners, riders and equine handlers for the generation of health parameter measurements at rest and during movement as the harness and saddle pad can be safely and securely anchored around the animal's torso with the sensors and wires protected from any potential damage by a rider or the mobility of the animal.

The system of the invention is suitable for use with performance animals such as horses, camels, dromedaries, greyhounds and the like where performance and health are of concern and value to owners and trainers etc. However, the system is particularly suitable for use with racehorses and performance horses. No extra padding or other additional items are required and, due to the automatic and correct placement of the ECG electrodes and other sensors by the harness and saddle pad, no additional set up time or training is required so that the system can be easily and quickly employed by an unskilled user.

As indicated above, the cassette type electronics assembly is also transferable between the harness and the saddle pad of the system so that health and performance analyses can be performed pre- post- or during exercise and the data retrieved and communicated as required from the cassette type electronics assembly.

The materials of the system are selected for optimal performance of the sensors (e.g. medical grade electrodes) and protection of the animal. The materials reduce interference in for example ECG signal/readings. Similarly, the wiring employed is formed from appropriate materials and insulated to prevent interference. More particularly, by employing natural fibres such as cotton where possible, the materials of the system do not contain high levels of interference enhancing fibres. The materials chosen allow for stretch and movement whilst also ensuring the wiring is protected from movement, sweat and over compression. As indicated above, a tough outer layer is also employed with incorporated stretch capability to ensure that the system wiring is protected sufficiently in the harsh environment to which it will be placed.

### BRIEF DESCRIPTION OF THE DRAWINGS

The invention will now be described, by way of example only, with reference to the accompanying drawings in which:

FIG. 1 is a front and side image of a horse fitted with a harness of the invention;

FIG. 2 is a front image of the horse and harness of FIG. 1;

FIG. 3 is a side image of the harness;

FIG. 4 is a top/front plan view of the harness showing the rib portion and chest portion with integrated wiring and the centrally located holster;

FIG. 5 is a bottom/rear plan view of the harness of FIG. 4 showing the ECG sensors on the rib portion and chest portion;

FIG. 6 is a schematic drawing of the integrated wiring concealed in the harness;

FIG. 7 is a cross-sectional view through a portion of the harness showing the air mesh inner layer in which the wiring is concealed, an animal contacting layer and an environmentally tough outer fabric layer;

FIG. 8 is a cross-sectional view through the harness at an ECG sensor with the wiring connected to the sensor via a crimped connector and an ECG sensor connector;

FIG. 9 is a cross-sectional view of the harness as shown in FIG. 8 with the ECG sensor connected to the ECG sensor connector;

FIG. 10 are bottom and top view of the wither pad;

FIG. 11 is an exploded view of the holster;

FIG. 12 is a perspective view from above and one side of the first part of a two part nine pin magnetic connector for connecting the wiring to the electronic assembly;

FIG. 13 is a perspective view from above and one side of the second part of the two part nine pin magnetic connector of FIG. 12;

FIG. 14 is a side perspective of a horse with a saddle pad of the system of the invention which is complementary with the harness of FIGS. 1 to 13 placed on the horse with two of three ECG sensors, associated concealed integrated wiring visible for illustrative purposes extending to an electronic assembly (in an external holster) on a sixth rib radius portion of a first side apron of the saddle pad with a single electronic assembly being interchangeable between the harness and the saddle pad;

FIG. 15 is a side perspective view of the horse of FIG. 1 with a saddle placed over the saddle pad to secure the saddle pad in place on the horse with the girth of the saddle which is coincident with the sixth rib radius portions of the saddle pad;

FIG. 16 is an enlarged top plan view of the saddle pad of FIGS. 14 and 15 laid flat to show the two side aprons of the saddle pad;

FIG. 17 is a side view of the saddle pad of FIG. 16 in the use position showing the first side apron of the saddle pad as illustrated in FIG. 14;

FIG. 18 is a perspective view from above of the top side of a dual use self-contained, unitary and removable cassette-type electronic assembly of the system of the invention for insertion in the complementary holster;

FIG. 19 is a perspective from above of the bottom side of the cassette-type electronic assembly of FIG. 18;

FIG. 20 is a perspective view from above and one side of the complementary holster;

FIG. 21 is a side perspective view of a second embodiment of a saddle pad of the system of the invention on a horse (with a rider shown mounted on the horse for illustrative purposes) with the electronic assembly and associated holster positioned rearwardly of the rider and the wiring visible also for illustrative purposes;

FIG. 22 is a side perspective view of the horse and saddle pad of FIG. 21 with the rider removed and the ECG and associated shortened wiring between the electrodes and the electronic assembly visible for illustrative purposes;

FIG. 23 is an enlarged side view of the side apron of the saddle pad with holster and electronic assembly of FIG. 22 showing electrode alignment with the girth strap and sixth rib radius portion of the horse;

FIG. 24 is an exploded view of the structural material/fabric layers of the saddle pad;

FIG. 25 is a side view of the structural material/fabric layers of the saddle pad;

FIG. 26 is a plan view of the side apron of FIG. 23 with the electrode compressors in the form of compression foam blocks for compressing/urging the ECG electrodes against the horse's skin visible;

FIG. 27 is a plan view of the elongate generally rectangular ECG electrodes on the side apron compressed by the electrode compressors of FIG. 26;

FIG. 28 is an enlarged view of the ECG electrodes of FIG. 27 attached to the electronic assembly by short wiring to minimise interference/noise;

FIG. 29 is an enlarged plan view of the elongate ECG electrodes removed from the saddle pad with the ECG electrodes having a rectangular major portion and a leg portion to configured to form an L-shape for optimal signal recording, and

FIG. 30 is a flow diagram of the method employed with the chest harness and/or the saddle pad of the system of the invention to link health data to a horse microchip.

### DETAILED DESCRIPTION OF THE INVENTION

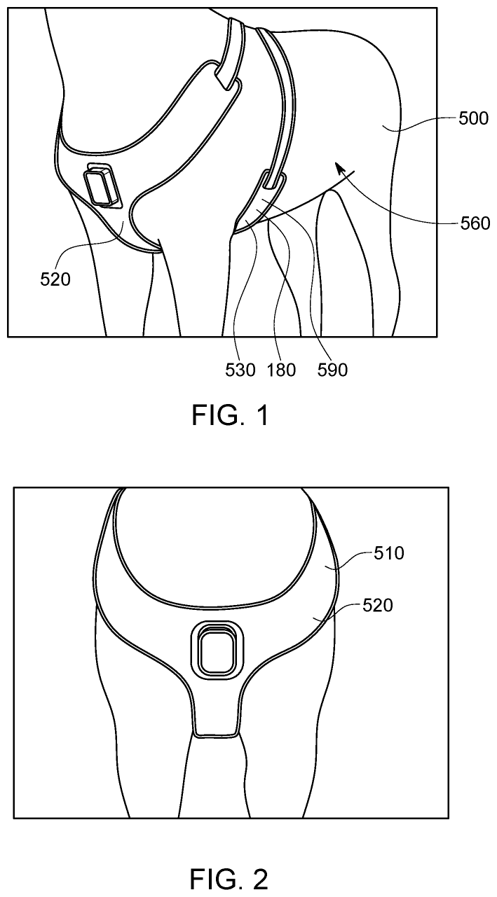

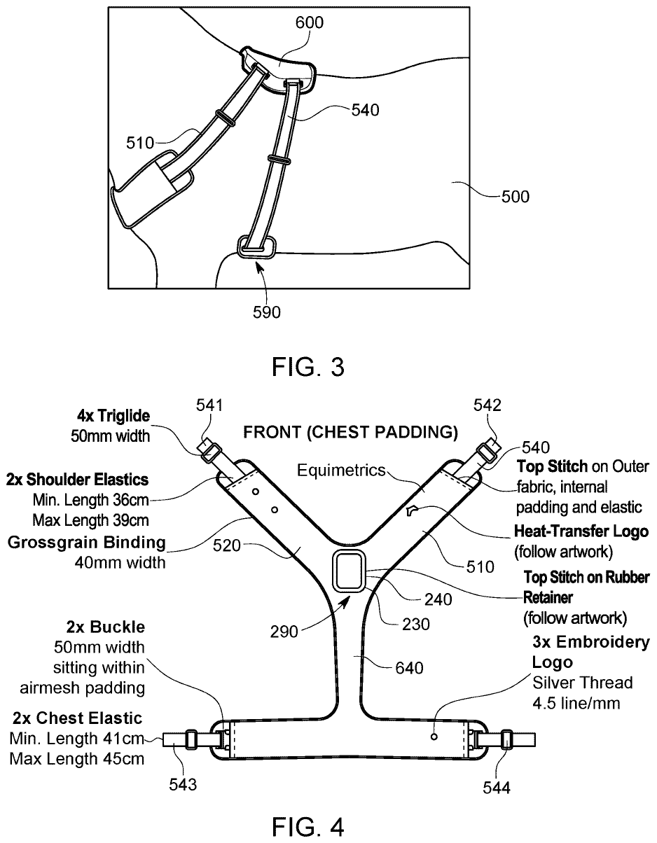

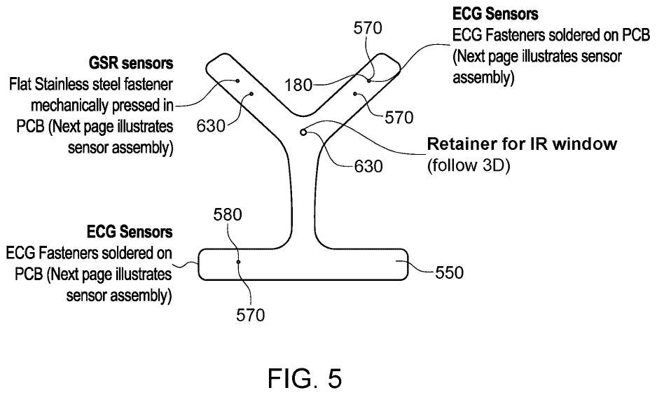

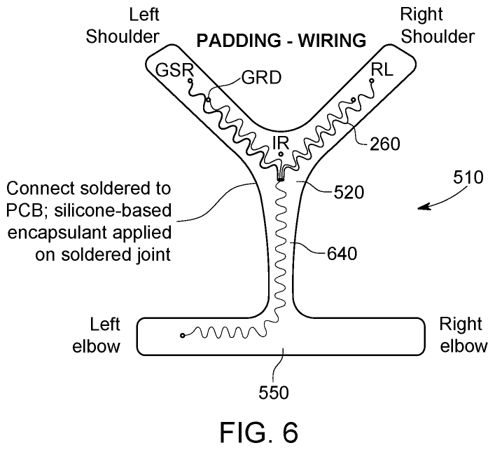

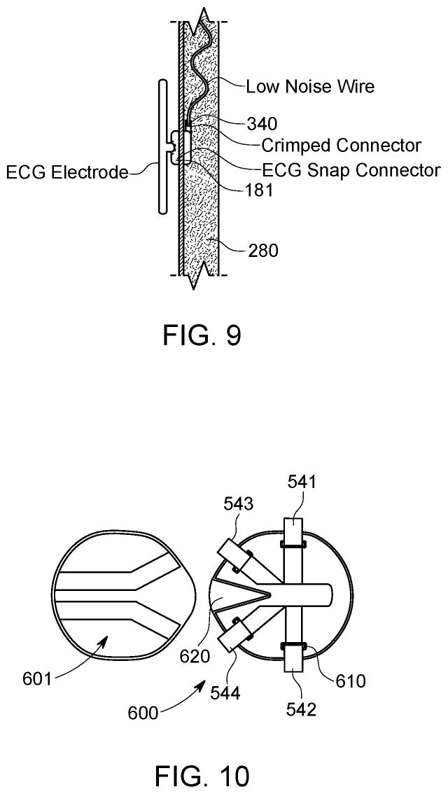

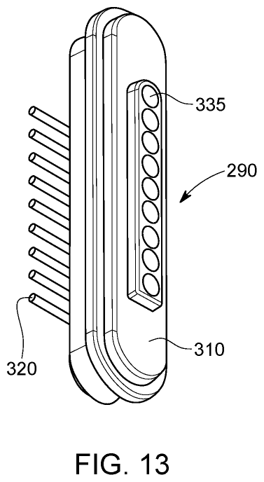

As shown initially in FIGS. 1 to 13 and 18 to 20, a chest harness of an animal health monitoring system of the invention for use on an animal such as a horse 500 is generally indicated by the reference numeral 510 and is made up of a chest portion 520 (which is generally Y-shaped when not in use), which can be in the form of a chest pad 520, mountable at the chest of the horse 500 and straps 540 for supporting the chest portion 520 on the horse 510. The chest harness 510 is also provided with a rib portion 550, which can be in the form of a rib pad 550, attached to the chest portion 520 by the straps 540 configured for positioning at the ribs 560 of the horse 500. The rib portion 550 is provided with a sixth rib radius locus 570 for receiving an ECG sensor 580 and is configured to locate the ECG sensor 580 adjacent a sixth rib radius 590 of the horse 510.

The harness 510 is further provided with a wither pad 600 connected to the chest portion 520 and the rib portion by the straps 540 to anchor the chest harness 510 in place on the horse 500. As shown in the drawings, the straps 540 are made up of two adjustable chest portion straps 541,542 which extend between the free ends of the elongate chest portion 520 and the wither pad 600 and two rib portion straps 543,544 which extend between the elongate rib portion 550 and the wither pad 600. The wither pad 600 is an ergonomic wither pad 600 shaped and contoured in accordance with the anatomy of the horse 500 to reduce the risk of pressure sores on the wither. More particularly, the wither pad 600 is provided with slot-like strap mountings and buckles 610 shaped and configured on its underside as indicated by the reference numeral 601 in accordance with the shoulder anatomy of the horse 500. In particular, the slot-like strap mountings and buckles 610 are oriented at 90 degree angles to align with the natural shoulder shape of the horse. To reduce the risk of pressure sores.

The wither pad 600 also has a spinal clearance or spine recess 620 to accommodate the spine of the horse 500 and is provided with a grip fabric on its animal contacting side.

The harness 510 is further supported in place by a central panel 640 which extends between the rib portion 550 and the chest portion 530 and is configured to secure around the girth area ensuring a safe and secure placement which does not interfere with movement.

As shown in the drawings, an ECG sensor 180 is located at the sixth rib radius locus 570 which can be detachably mounted on the sixth rib radius locus 570 with a connector 181 such as a snap connector 181. The chest portion 520 can also comprise sixth rib radius loci 570 for receiving ECG sensors 180 as required—e.g. the rib portion 550 can receive a first ECG sensor 180 and the chest portion can receive second and third ECG sensors to attain a cross section signal of the heart. The ECG sensors can comprise ECG electrodes 180 mounted on an external animal contacting surface of the sixth rib radius loci 570.

As indicated by the reference numeral 630, the chest harness 510 is further provided additional sensors as required for monitoring the health and wellbeing of the horse 500 which can include any or all of temperature, respiration, moisture and movement sensors which can be positioned on the rib portion 550 and/or the chest portion 520 as required.

The chest harness 230 has an electronic assembly 230 communicable with the ECG sensor mounted at a mounting 240 on the chest portion 520 and integrated wiring 60 concealed within the rib portion 550 and the chest portion 520 extends between the sensors and the electronic assembly 230. The wiring 260 is threaded between the sensors and the electronic assembly 230 in an undulating, coiled or zig-zag pattern

Importantly, the wiring 260 is concealed within an inner layer 270 of the chest portion 520 and rib portion 530. A particularly suitable material for the inner layer 270 is air mesh fabric 280 through which the wiring 260 can be threaded. More particularly, the wiring 260 is threaded between the sensors and the electronic assembly 230 through the air mesh fabric 280 in the undulating, coiled or zig-zag pattern described above to minimise mechanical stress on the wiring 260.

The wiring 260 is connected to the electronic assembly 230 via a magnetic connector 290 at the mounting 240 and the magnetic connector 290 can be a fluid tight nine pin magnetic connector 290. The magnetic connector 290 is a two-part detachable magnetic connector 290 at the holster 250. Both the magnetic connector 290 and the holster 250 ensure that the connections formed between the wiring 260 and the electronic assembly 230 are fluid-tight. As discussed in more detail below, the two-part magnetic connector 290 is made up of a male part 300 and a female part 310 both provided with nine pins 320 for transmitting signals and data between the ECG electrodes 180,190,200 and the electronic assembly 230. The male part 300 is provided with magnetic contacts 330 which are contactable with complementary magnetic contacts 335 on the female part 310.

The magnetic contacts 330,335 are gold plated to reduce the risk of corrosion from the elements/harsh environment and animal sweat.

The magnetic connection ensures correct mating of the male and female parts 300,310 in the holster 250 (which can be unforgiving should the mating of the electronic assembly to the holster on the saddle pad 10 be incorrect. This unforgiving nature is due to the safety and security required to keep the electronic assembly 230 safely in situ.

The mounting comprises a holster 250 for housing the electronic assembly 230 which is centrally located on the chest portion 520.

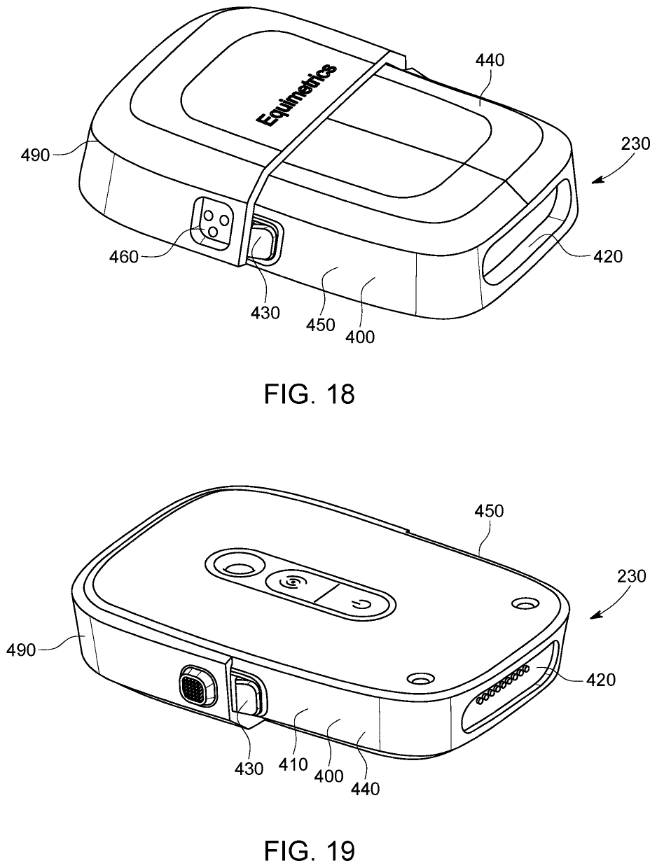

As shown particularly in FIGS. 18 to 20, the electronic assembly 230 comprises a self-contained and removable cassette 400 electronic assembly complementary in size and shape with the holster 250. As shown in the drawings, the cassette 400 comprises a male 300 or female 310 part of the magnetic connector 290 and the holster 250 comprises a complementary male 300 or female 310 part of the magnetic connector 290. The wiring 260 is formed from low-noise wires connected to the sensors by a crimped connector 340.

As indicated above and as shown particularly in FIGS. 18 to 20, in the present embodiment, the electronic assembly 230 is a self-contained, unitary and removable cassette-type electronic assembly 230 which is insertable in the holster 250 which is sized, shaped and contoured to removably receive the electronic assembly. More particularly, the electronic assembly 230 is in the form of a generally rounded rectangular cassette 400 having a fluid-tight housing 410 provided with an electronic connector opening 420 at one end for facilitating electronic communication with electronics securely contained within the housing 410. The housing 410 is also provided with side-fasteners or clips 430 on its sidewalls 440,450 which are button 460 actuatable to secure the cassette to the complementary holster 250 shown in FIG. 12.

The magnetic connector 290 has a fluid-tight/waterproof grading (in the present embodiment an IP 67 rating) to limit the risk of moisture ingress to either the electronic assembly 230 or the holster 250. This also reduces the risk of water ingress when the devices are not connected and vulnerable.

The magnetic connector 290 also ensures perfect conductivity for the ECG signal extraction and secure fitting of the electronic assembly 230 at speed.

The chest portion 520 and the rib portion 550 are provide with an environmentally tough fabric outer layer for durability.

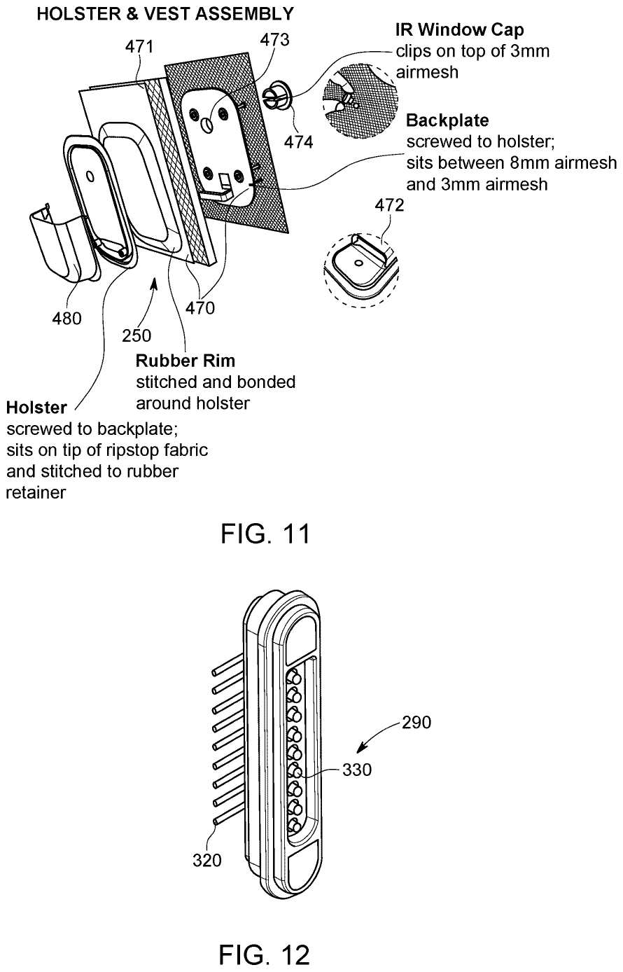

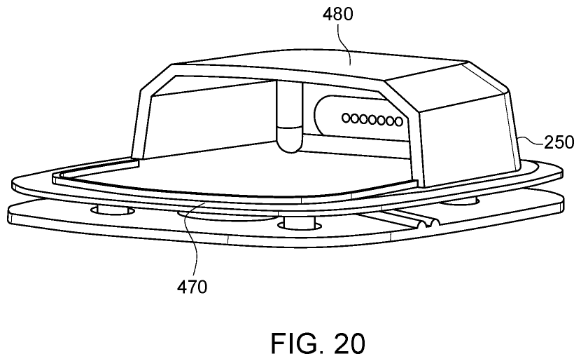

FIGS. 11 and 20 show an exploded view of the holster. As shown in drawings, the holster 250 is made up of a base plate 470 provided with a pocket 480 for receiving the cassette 400 in a tight fit. The base plate 470 can be formed with a panel 471 and a rubber rim 472 while the back plate 470 is provided with an IR window 473 for an IR temperature sensor which can be closed with a cap 474. In the present embodiment, the male part 300 of the magnetic connector 290 is located on the cassette 400 at the connector opening 420 and the female part 310 of the magnetic connector 290 is contained within the pocket 480. The cassette 400 is further provided with a protective cover 490. The cassette 400 is therefore quickly insertable in and removable from the complementary holster 250 as required.

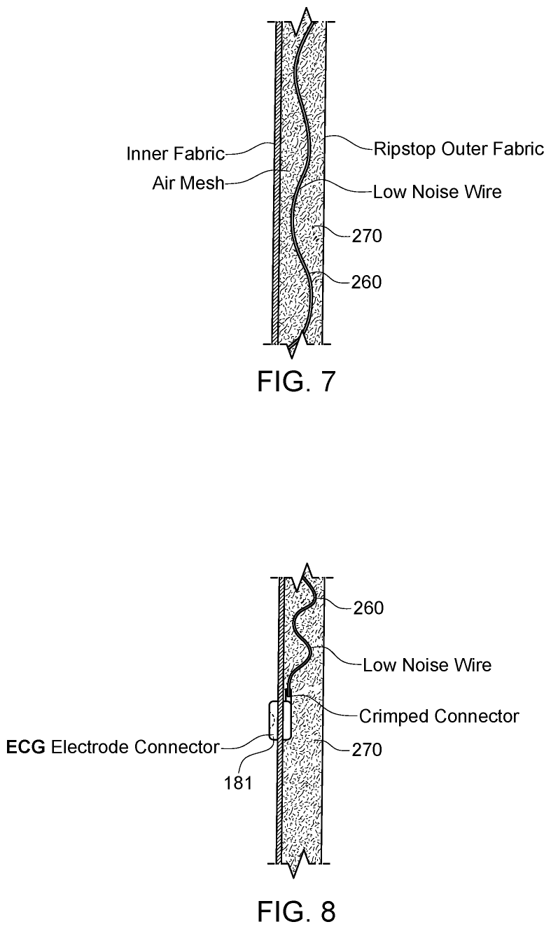

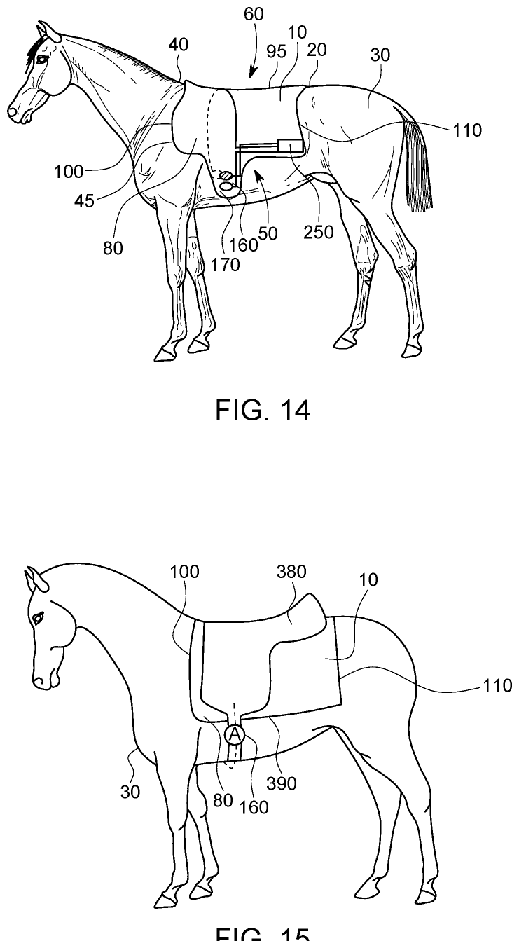

As shown in FIGS. 14 to 20, and also with reference to FIGS. 7 to 9, 12 to 13, a saddle pad of the system of the invention for placement on the back 20 of a horse 30 is generally indicated by the reference numeral 10. The saddle pad 10 is sized, shaped and contoured to extend rearwards from the withers 40 and the shoulders 45 of the horse 30 and to extend downwards over the left ribs 50 and right ribs 60 of the horse 30. More particularly, the saddle pad is made up of a panel-like first or left apron 80 for extending over the left ribs 50 and an opposite panel-like second or right apron 90 for extending over the rights ribs 60. The first and second aprons 80,90 meet at central spine portion 95 and define a saddle pad front end 100 for placing towards the withers 40, a saddle pad rear end 110, a first/left side edge 81 at the first apron 80 and a second/right side edge 91 at the second apron 90. The front end 100 is shaped and contoured to define left and right shoulder panels 120,130 and a central withers panel 135 at the central spine portion 95.

Importantly, the left side edge 81 of the left apron 80 is shaped, sized and contoured to define a left sixth rib radius portion 140 for receiving an ECG sensor 180. The sixth rib radius portion 140 is positioned and configured on the saddle pad 10 to locate over a left sixth rib radius 160 of the horse 30. Similarly, right side edge 91 of the right apron 90 is shaped, sized and contoured to define a right sixth rib radius portion 150 for receiving an ECG sensor 190. The right sixth rib radius portion 150 is also positioned and configured on the saddle pad 10 to locate over a right sixth rib radius of the horse 30. Each of the left and right sixth rib radius portions 140, 150 are in the form of flaps 170 which depend from the respective side edges 81,91 of the left and right aprons 80,90.

The left and right sixth rib radius portions 140, 150 therefore accurately, automatically and reproducibly locate the ECGH sensors at the sixth rib radius 160 of the horse for optimal ECG readings.

In the present embodiment, the first and second ECG sensors 180,190 are ECG electrodes 180,190 and the left sixth rib radius portion 140 is further provided with a third ECG sensor electrode 200. Each of the ECG electrodes 180,190,200 is located at an animal facing surface 210,220 respectively of the left and right sixth rib radius portions 140,150. The ECG electrodes 180, 190,200 are detachably mounted to the animal facing surface 210,220 via sensor connectors 350 so that the ECG electrodes 180,190,200 can be easily replaced as required. Suitable sensor connectors 350 are snap connectors 350.

Accordingly, in this arrangement, two electrodes 180,190 on the left and right sixth rib radius portions 140,150 achieve a cross section reading of the horse's heart at the 6^{th} rib radius 160 and the third electrode 200 disposed above the first electrode 180 stabilises the signal by serving as a reference grounding lead.

The ECG electrodes 180,190,200 communicate with an electronic assembly 230 mounted on the saddle pad 10 towards the rear end 110 at an electronic assembly mounting 240 so that the electronic assembly 230 does not interfere with a rider. In the present embodiment, the electronic assembly 230 is contained within a holster 250 at the mounting 240 which is located on the left sixth rib radius portion 140. The saddle pad 10 is provided with integrated wiring 260 which is concealed within the saddle pad 10 and extends between the electrodes 180,190,200 and the electronic assembly 230 in the holster 250. The wiring 260 does not therefore present a safety hazard.

In the present embodiment, the wiring 260 is contained within an inner layer 270 of the saddle pad 10 and more particularly within inner layers 270 of the left and right aprons 80,90 and spine portion 95. A particularly suitable material for the inner layer 270 is air mesh fabric 280 through which the wiring 260 can be threaded. More particularly, the wiring 260 is threaded between the ECG electrodes 180,190,200 and the electronic assembly 230 through the air mesh fabric 280 in an undulating, coiled or zig-zag pattern to minimise mechanical stress on the wiring 260.

The wiring 260 is connected to the electronic assembly 230 via a two-part detachable magnetic connector 290 at the holster 250. Both the magnetic connector 290 and the holster 250 ensure that the connections formed between the wiring 260 and the electronic assembly 230 are fluid-tight. The two-part magnetic connector 290 is made up of a male part 300 and a female part 310 both provided with nine pins 320 for transmitting signals and data between the ECG electrodes 180,190,200 and the electronic assembly 230. The male part 300 is provided with magnetic contacts 330 which are contactable with complementary magnetic contacts 335 on the female part 310.

The magnetic contacts 330,335 are gold plated to reduce the risk of corrosion from the elements/harsh environment and animal sweat. Three of the nine pins 320 are for the ECG electrodes 180,190,200, two for galvanic skin response (bioimpedence), two for data and two for power.

The magnetic connection ensures correct mating of the male and female parts 300,310 in the holster 250 (which can be unforgiving should the mating of the electronic assembly to the holster on the saddle pad 10 be incorrect. This unforgiving nature is due to the safety and security required to keep the electronic assembly 230 safely in situ whilst the horse 30 moves/at speed etc.

As with the chest harness 510 and as shown particularly in FIGS. 10 to 12, in the present embodiment, the electronic assembly 230 is a self-contained, unitary and removable cassette-type electronic assembly 230 which is insertable in the holster 250 which is sized, shaped and contoured to removably receive the electronic assembly. More particularly, the electronic assembly 230 is in the form of a generally rounded rectangular cassette 400, which is interchangeable between the chest harness 510 and the saddle pad 10, having a fluid-tight housing 410 provided with an electronic connector opening 420 at one end for facilitating electronic communication with electronics securely contained within the housing 410. The housing 410 is also provided with side-fasteners or clips 430 on its sidewalls 440,450 which are button 460 actuatable to secure the cassette to the complementary holster 250 shown in FIG. 12. The holster 250 is made up of a base plate 470 provided with a pocket 480 for receiving the cassette 400 in a tight fit. In the present embodiment, the male part 300 of the magnetic connector 290 is located on the cassette 400 at the connector opening 420 and the female part 310 of the magnetic connector 290 is contained within the pocket 480. The cassette 400 is further provided with a protective cover 490. The cassette 400 is therefore quickly insertable in and removable from the complementary holster 250 as required.

The magnetic connector 290 has a fluid-tight/waterproof grading (in the present embodiment an IP 67 rating) to limit the risk of moisture ingress to either the electronic assembly 230 or the holster 250. This also reduces the risk of water ingress when the devices are not connected and vulnerable.

The magnetic connector 290 also ensures perfect conductivity for the ECG signal extraction and secure fitting of the electronic assembly 230 at speed.

In order to further enhance to ECG signal from the ECG electrodes 180, 190,200, the wiring 260 is formed from low-noise wires and is connected to the ECG sensors 180,190,200 by crimped connectors 340.

As indicated above, the inner layer 270 of the saddle pad can be formed from an air mesh fabric 280 while, in order to ensure robust performance of the saddle pad 10, the saddle pad 10 can be provided with a ripstop fabric outer layer 360 and an inner fabric animal facing layer 370 so that air mesh fabric 280 is sandwiched between the outer layer 260 and the animal facing layer 370.

In use, the saddle pad 10 is placed on the horse 30 as shown particularly in FIG. 15 in a three step process. Firstly, the saddle pad 10 is placed over the back 20 of the horse 30 as shown in FIG. 15. Due to the ECG electrodes 180,190,200 being positioned at the left and right sixth rib radius portions 140,150, the ECG electrodes 180, 190,200 are automatically located at the sixth rib radii 160 of the horse 30. Thirdly, a saddle 380 is placed over the saddle pad 10 so that the saddle pad is held in place by the girth 390 of the saddle 380 as the left and right sixth rib radius portions 140, 150 are configured to be coincident with the girth 390 when placed on the horse 30. As a result, the left and right sixth rib radius portions 140,150 are sandwiched between the sixth rib radii 60 of the horse 30 and the girth 390 to securely hold the left and right sixth rib radius portions 140,150 and associated ECG electrodes in position at the sixth rib radii 60 during exercise.

Signals generated by the ECG electrodes 180,190,200 are captured by the electronic assembly 230 via the concealed wiring 260 and the magnetic connector 290 encapsulated in the integrated holster 250. In one embodiment, the electronic assembly 230 can communicate directly by Bluetooth (Trade Mark) or other enabled communication with an App and then forward data to an analytical structure and subsequently through a series of algorithms to the cloud. A final output is then displayed on a device of choice which can be a hand held device, laptop or similar display device. The ECG data can be displayed in graph format e.g. normal or previous ECG traces versus the current extraction. This enables a direct observation for vets or trainers as to any changes or abnormalities in the animal's coronary condition under stress.

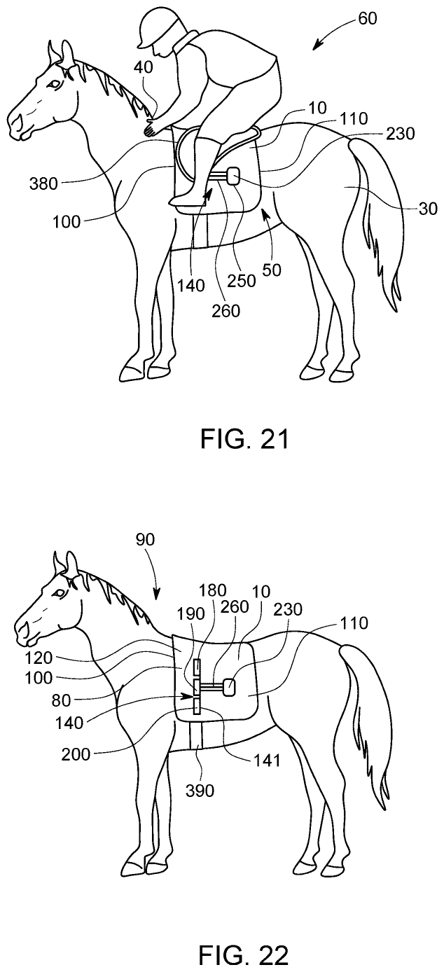

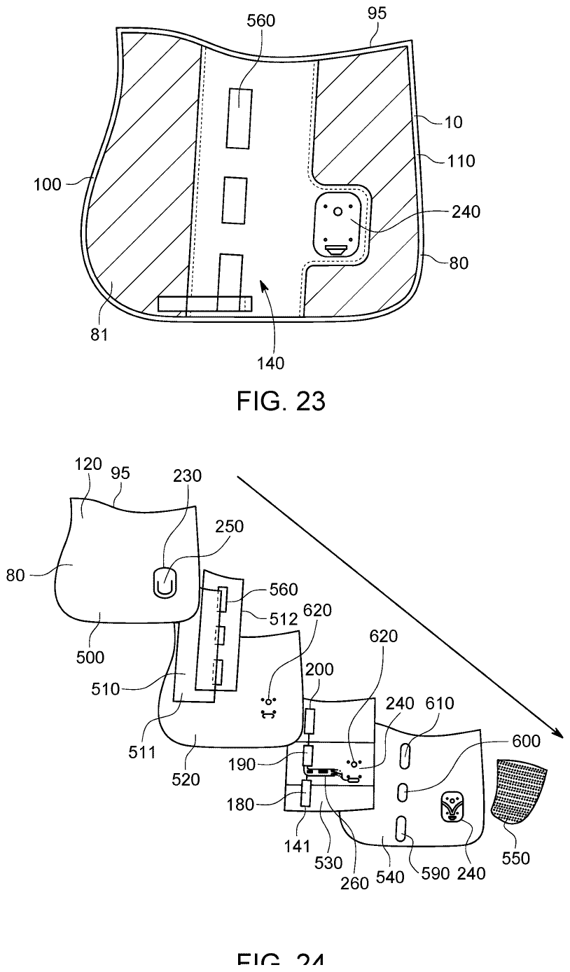

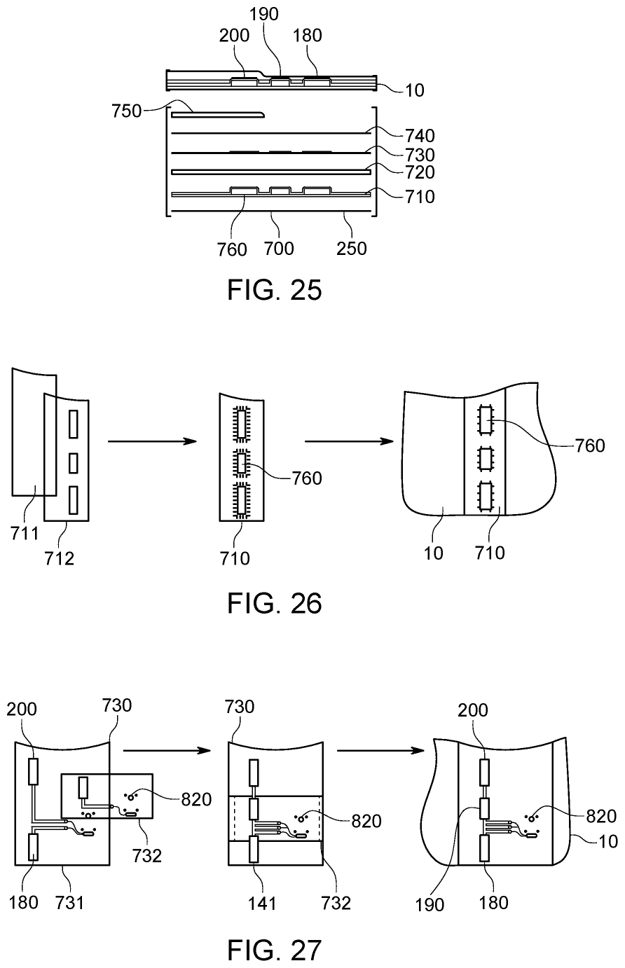

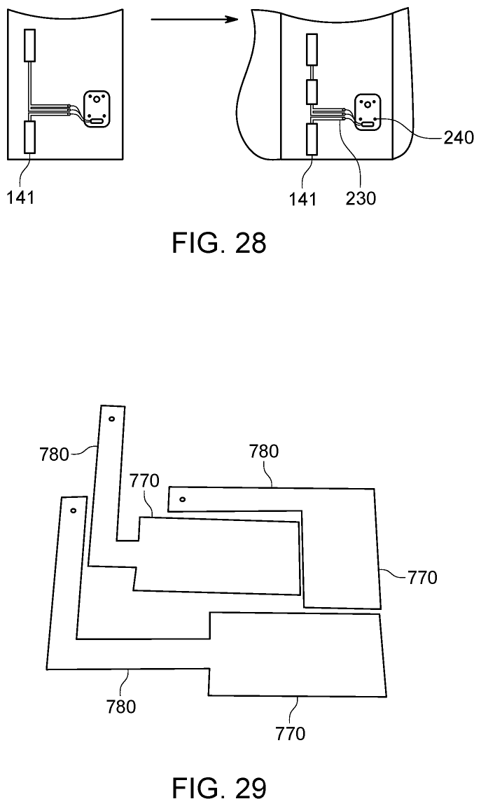

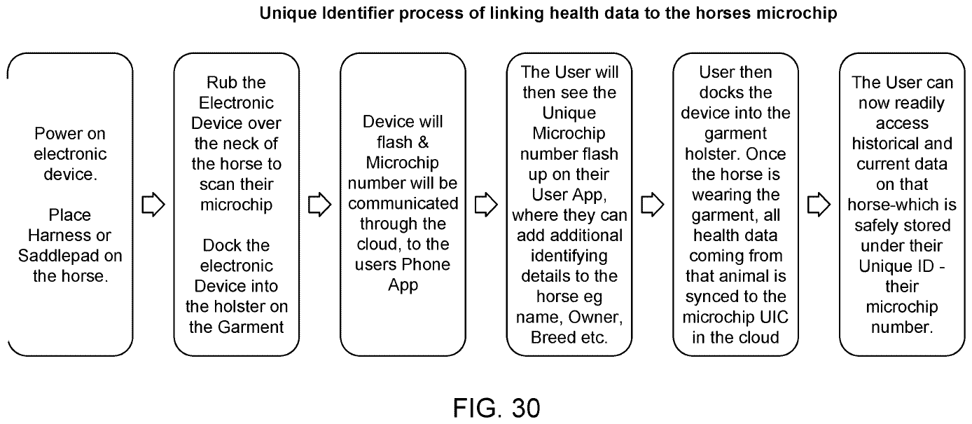

FIGS. 21 to 30 show a second embodiment of a saddle pad 10 suitable for use in the system of the invention in combination with the harness 510 on a horse broadly similar to the saddle pad of FIGS. 14 to 17 and like numerals indicate like parts. However, in the present embodiment, the sixth rib radius portion(s) 140,150 are not located at depending flaps 170 but are defined on the side apron 80 at the sixth rib radius portion 80 along a line which is configured to be contiguous with the girth 390 of a saddle.

As shown in the drawings, the saddle pad 10 is provided with three ECG sensors 180,190,200 as previously described. However, in the present embodiment, the ECG sensors 180,90,200 are located on the left apron 80 at ECG loci 141 disposed along a line which is configured and spaced between the front end 100 and rear end 110 of the saddle pad 10 to align with the saddle girth 390 in use. The ECG electrodes are in electronic communication with the electronic assembly 230 which is detachably mounted in a holster 250 on the left apron 80 disposed towards the rear end 110 of the apron 80 so that electronic assembly 230 is disposed behind a rider's limbs in use to minimise impacts and interferences at the electronic assembly 230.

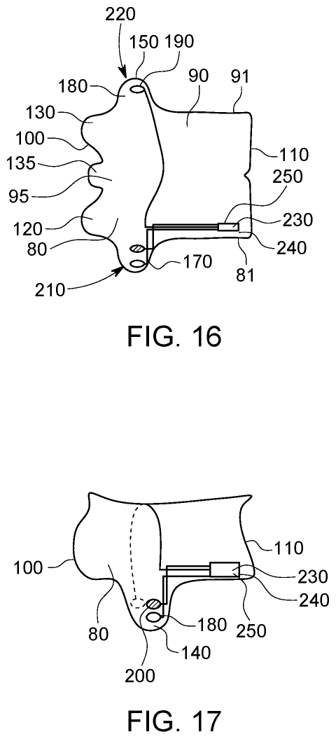

As shown particularly in FIGS. 16 and 17, in the present embodiment, the side apron 80 of the saddle pad 10 is also a multi-layered side apron 80 and, from the outside to the inside, is made up of an outer layer 700 with the holster 250, an ECG sensor compressor layer 710 for compressing the ECG sensors 180, 190,200 against the skin, a padding layer 720, an ECG sensor carrying layer 730, an inner layer 740 and a non-slip pad on the inner layer to prevent slippage. The wiring 260 is therefore concealed within the saddle pad 10 as before. The materials of the various layers are formed from natural materials as much as possible to minimise noise. For example, the outer layer can be cotton, the ECG sensor compressor layer 710 can include ECG compressors 760 in the form of sensor compressor blocks 760 for each ECG sensor 180,190,200, a natural padding layer 720 such as cotton, an ECG sensor carrier layer 730, an inner layer 740 also for example cotton and a non-slip pad 750 on the inner layer 740. The use of natural materials such as cotton minimizes noise and static interference while the use of a non-slip pad 750 keeps the saddle pad 10 in place and prevents slippage. A suitable non-slip pad 750 is formed from a nonslip grip having a thickness of about 1-5 mm. A suitable cotton material is medium weight cotton twill. The cotton materials also prevent unnecessary sweating. The padding layer 720 can be a soft material having a thickness of 5-10 mm.

In the present embodiment, the ECG sensor compressor layer 710 has three compressor blocks 760 arranged along a line spatially corresponding with the three ECG sensors 180, 190,200 so that the compressor blocks 760 are also configured to locate the compressor blocks 760 and urge or compress the ECG sensors 180,190,200 against the skin, at the ECG loci adjacent the sixth rib radius of the animal. The ECG compressor layer 710 is a double cotton twill fabric layer having first and second sub-layers 711,712 between which the compressor blocks 760 are encased. The cotton of the ECG sensor compressor layer 710 can be a lightweight cotton twill having a thickness of 1-5 mm. The sub-layers 711,712 are top stitched together to secure the foam blocks 760 in place and the compressor layer 710 is in turn stitched to the outer layer 700. The compressor blocks 760 can be formed from a closed cell foam material of suitable hardness and a size corresponding with that of the electrodes 180, 190,200 to provide the required structure for maximum skin contact with and pressure on the ECG electrodes 180,190,200. The foam compressor blocks 760 are sized to match the ECG electrodes 180,190,200 and can be formed from a firm medium or high-density foam (e.g. HD 30) having a thickness ranging from 5-15 mm e.g. 10 mm.

As shown particularly in FIGS. 24 and 27 to 29, the sensor carrier layer 730 is stitched to the inner layer 740 and is provided with the three ECG electrodes 180,190,200 at ECG loci 141 disposed along a line which is configured and spaced to align with the saddle girth 390 and the sixth rib radius portion of the animal in the assembled saddle pad 10. Two of the three ECG electrodes 180,190,200 provide a cross sectional reading of the heart while the third electrode is a grounding electrode. This is facilitated by arranging the ECG electrodes 180, 190,200 in a vertical sequence, as aligned with the girth, so that the electrodes 180, 190,200 read the top and bottom of the heart i.e. the ECG electrodes provide a functional cross sectional view of the heart for accurate ECG readings As with the ECG compressor layer 710, the sensor carrier layer 730 is a double layer material having a first large sub-carrier layer 731 and a second smaller sub-carrier layer 732. The ECG electrodes 180, 200 are bonded to the large sub-carrier layer 731 and the ECG sensor 190 is bonded to the small sub-carrier layer 532. The bonding of the ECG to the sensor carrier layer 730 also assists in optimising skin contact. The ECG electrodes 180,190,200 are suitably carbon silicone rubber electrodes having an optimal shape, size and design for enhanced ECG readings. As shown particularly in FIG. 29, the ECG electrodes 180,190,200 are shaped and configured to form an L-shape for optimal signal recording. More particularly, the ECG electrodes 180,190,200 each have a rectangular major signal receiving portion 770 and a leg portion 780 for connecting and aligning the electrodes 180,190,200 along the sixth rib radius portion in use. The ECG electrodes 180,190,200 also have an increased surface area to cover a greater area of the animal's rib cage. For example, the ECG electrodes 180,190,200 can be made up of two (RA/LA) electrodes having an area of about 110×40 mm and a noise reducing RLD electrode having an area of about 80×40 mm. The increased size of the ECG electrodes 180,190,200 also ensures that length of wiring 260 required between the electronic assembly 230 and the electrodes 180,190,200 is reduced to further reduce noise and enhance the ECG signal. More particularly, the L-shape of the electrodes 180,190,200 provide ease of manufacture and overall product design and tooling of the saddle pad as tooling a rectangular electrode is more straightforward than a circular design. In addition, the L-shape facilitates an increase in surface area while the leg portion 580 allows for a reduction in wire length-all with the aim of reducing triboelectric noise for enhanced signal strength.

As shown in the drawings, the ECG loci 141,151 and hence the ECG electrodes 180,190,200 are also configured to be located towards the upper side of the sixth rib radius portion rather than on the lower side to reduce noise, improve the signal and eliminate any rider movement interferences. The sensor carrier layer is also provided with an electronic assembly mounting 240 as previously described in communication with the ECG electrodes 180,190,200 via the wiring 260. The ECG electrodes 180,190,200 are automatically and optimally located at the sixth rib radii 160 of the horse 30. In use, a saddle 380 is placed over the saddle pad 10 as before so that the saddle pad 10 is held in place by the saddle 380 itself and the girth 390 of the saddle 380 as the left and right sixth rib radius portions 140,150 are configured to be coincident with the girth 390 when placed on the horse 30. However, in the present embodiment, the wiring 260 is configured to extend rearwardly from the electronic assembly 230 away from the saddle 380 so that the wiring 260 is situated behind a rider's leg in use and is not compressed by the saddle 10 thus further reducing signal interferences.

The inner layer 740 is shaped and sized to correspond with the outer layer 700 to form the left apron 80 of the saddle pad 10. However, the inner layer 740 is provided with three ECG electrode window-like openings 790,800,810 for receiving the ECG electrodes 180,190,200 respectively so that the ECG electrodes 180, 190,200 are fully exposed to the horse's skin via the window-like openings 790,800,810 i.e. the ECG electrodes 180,190,200 are in full contact with the skin of the animal for optimal signal pick-up and generation. The inner layer 740 can also include a portion of the electronic assembly mounting 240.

An electronic assembly 230 in the holster 250 can communicate with the wiring 230 via electronic communication ports 820 provided in the various layers of the saddle pad 10.

In the present embodiment, the mounting 240 for the electronic assembly 230 and the holster 250 is disposed forwardly of the saddle pad rear end 110 towards the saddle pad front end 100 and the withers 40 i.e. towards the ECG loci 141 at the sixth rib radius portion 140 of the side apron and hence the ECG electrodes 180,190,200 without interfering with a rider's legs to be as proximate to the ECG electrodes 180,190,200 as possible thereby reducing the length of the wiring 260 to reduce triboelectric noise and improve the ECG signal.

In another embodiment of the invention, the saddle pad 10 can incorporate other sensors to examine other health data in exercise. For example, the saddle pad 10 can include GPS, gyroscopes, accelerometers and temperature sensing technology to collect motion analysis to measure speed, distance, location, altitude, stride length, effort, stride cadence, HR analysis, time, speed, effort, response to topography and body temperature etc.

The saddle pad 10 of the invention can programmed with software as required for use with microchipped horses. In use, the saddle pad 10 of FIGS. 21 to 29 can be employed as previously described. As shown in the drawings, the cassette 400 is first powered on and the harness 510 or the saddle pad 10 is placed on the horse 30. The cassette 400 is then rubbed over the neck of the horse 30 to scan for a microchip where present and is then docked in to the holster 250 on the harness 510 or the saddle pad 10. The cassette 400 then flashes and the microchip unique identifier code (UIC) is communicated via the cloud to a user's phone app or similar. The user can then see the horse's unique microchip number on their app and can add additional identifying details to the horse if desired such as hose name, owner, breed, etc. The user then docks the cassette 400 into the holster 250 so that all ECG data (and data from other sensors if present) from the animal is synchronised to the microchip UIC in the cloud. The user can access historical and current data on the horse which is safely stored under the UIC.

As indicated above, the health monitoring system of the invention can include the harness 510 of FIGS. 1 to 13 either alone or in combination with the saddle pad 10 of FIG. 14 to 20 or 21 to 29.

1. An animal health monitoring system comprising:
a chest harness having a chest portion mountable at the chest of the animal and a strap for supporting the chest portion on the animal,
and a rib portion attached to the chest portion configured for positioning at the ribs of the animal
wherein the rib portion and/or the chest portion comprises a sixth rib radius locus for receiving an ECG sensor configured to locate the ECG locus adjacent a sixth rib radius of the animal.

2. An animal health monitoring system as claimed in claim 1 wherein at least a first ECG sensor is located at the sixth rib radius locus.

3. An animal health monitoring system as claimed in claim 2 wherein the ECG electrode is detachably mounted on the sixth rib radius locus with a connector.

4. An animal health monitoring system as claimed in claim 2 wherein the rib portion and/or chest portion comprise oppositely disposed sixth rib radius loci for receiving first and second ECG sensors.

5. An animal health monitoring system as claimed in claim 4 wherein the rib portion and/or chest portion comprises a third ECG reference sensor at a sixth rib radius locus.

6. An animal health monitoring system as claimed in claim 2 wherein the ECG sensors comprise ECG electrodes mounted on an external animal contacting surface of the sixth rib radius loci.

7. An animal health monitoring system as claimed in claim 2 further comprising an electronic assembly communicable with the ECG sensor mounted at a mounting on the chest portion.

8. An animal health monitoring system as claimed in claim 7 further comprising integrated wiring concealed within the rib portion and the chest portion extending between the sensor and the electronic assembly.

9. An animal health monitoring system as claimed in claim 8 wherein the wiring is threaded between the sensor and the electronic assembly in an undulating, coiled or zig-zag pattern to minimise mechanical stress on the wiring.

10. An animal health monitoring system as claimed in claim 9 wherein the wiring is concealed within an inner layer of the chest portion and rib portion.

11. An animal health monitoring system as claimed in claim 10 wherein the inner layer comprises an air mesh fabric.

12. An animal health monitoring system as claimed in claim 8 wherein the wiring is connected to the electronic assembly via a magnetic connector at the mounting.

13. An animal health monitoring system as claimed in claim 12 wherein the magnetic connector is a nine pin magnetic connector.

14. An animal health monitoring system as claimed in claim 12 wherein the magnetic connector comprises a fluid-tight magnetic connector.

15. An animal health monitoring system as claimed in claim 8 wherein the mounting comprises a holster for housing the electronic assembly.

16. An animal health monitoring system as claimed in claim 15 wherein the holster is centrally located on the chest portion.

17. An animal health monitoring system as claimed in claim 16 wherein the electronic assembly comprises a self-contained and removable cassette electronic assembly complementary in size and shape with the holster.

18. An animal health monitoring system as claimed in claim 17 wherein the cassette comprises a male or female part of the magnetic connector and the holster comprises a complementary male or female part of the magnetic connector.

19. An animal health monitoring system as claimed in claim 8 wherein the wiring comprises low-noise wires.

20. An animal health monitoring system as claimed in claim 8 wherein the wiring is connected to the ECG sensor by a crimped connector.

21-35. (canceled)
